(19) United States
(12) Patent Application Publication
(10) Pub. No.: US 2023/0135216 A1
(43) Pub. Date: May 4, 2023

(54) INFORMATION PROCESSING SYSTEM AND INFORMATION PROCESSING DEVICE

(71) Applicant: SEIKO EPSON CORPORATION, Tokyo (JP)

(72) Inventor: Shuji UCHIDA, SHIOJIRI-SHI (JP)

(73) Assignee: SEIKO EPSON CORPORATION, Tokyo (JP)

(21) Appl. No.: 17/975,583

(22) Filed: Oct. 27, 2022

(30) Foreign Application Priority Data
Oct. 29, 2021 (JP) 2021-177660

(51) Int. Cl. A61B 5/107; A61B 5/11; A61B 5/00

(52) U.S. Cl. CPC A61B 5/1071; A61B 5/1123; A61B 5/6807; A61B 2562/0219

(57) ABSTRACT

An information processing system includes a measurement instrument and an information processing device, wherein the measurement instrument includes a data acquisition unit configured to acquire data about first shoes used by a user, the measurement instrument or the information processing device includes a measurement result acquisition unit configured to acquire a measurement result including at least a roll angle upon landing and a pitch angle upon landing, based on the data acquired by the data acquisition unit, and the information processing device includes a running motion determination unit configured to perform at least determination of a pronation score by comparison between the roll angle and a first threshold and determination of a landing score by comparison between the pitch angle and a second threshold and a suggestion determination unit configured to determine suggested shoes, based on at least the pronation score and landing score.

The present application is based on, and claims priority from JP Application Serial Number 2021-177660, filed Oct. 29, 2021, the disclosure of which is hereby incorporated by reference herein in its entirety.

### BACKGROUND

### 1. Technical Field

The present disclosure relates to an information processing system and an information processing device.

### 2. Related Art

People who do a running or the like use shoes.

How to run and the like may vary from person to person using shoes, so suitable shoes may vary from person to person.

A landing determination device described in JP-A-2019-181040 makes determination on landing of a user at the time of running at a tread mill, based on output of an accelerometer attached to the user. The landing determination device can decide suggested shoes, based on results of the determination including a landing mode and a level of impact upon landing (see JP-A-2019-181040).

However, in the related art, when shoes suitable for a user are suggested, the accuracy of the suggested shoes is insufficient in some respects.

For example, in the technology described in JP-A-2019-181040, the tread mill is used to analyze the running, so a result different from that of a case that actual running is analyzed may possibly be obtained through measurement. In the technology described in JP-A-2019-181040, a concrete example of a scheme for deciding suggested shoes from the running determination results is not disclosed, and a more concrete scheme for suggesting shoes suitable for the user is not considered.

### SUMMARY

An advantage of an aspect is an information processing system including a measurement instrument and an information processing device, wherein the measurement instrument includes a data acquisition unit configured to acquire data about first shoes used by a user, the measurement instrument or the information processing device includes a measurement result acquisition unit configured to acquire a measurement result including at least a roll angle upon landing and a pitch angle upon landing, based on the data acquired by the data acquisition unit, and the information processing device includes a running motion determination unit configured to perform at least determination of a pronation score by comparison between the roll angle and a first threshold and determination of a landing score by comparison between the pitch angle and a second threshold and a suggestion determination unit configured to determine suggested shoes, based on at least the pronation score and landing score.

An advantage of an aspect is an information processing device including a measurement result acquisition unit configured to acquire a measurement result including at least a roll angle upon landing and a pitch angle upon landing, based on an acquisition result of data about first shoes used by a user, a running motion determination unit configured to perform at least determination of a pronation score by comparison between the roll angle and a first threshold and determination of a landing score by comparison between the pitch angle and a second threshold, and a suggestion determination unit configured to determine suggested shoes, based on at least the pronation score and landing score.

### BRIEF DESCRIPTION OF THE DRAWINGS

FIG. 1 is a diagram illustrating a schematic configuration example of a first information processing system according to a first embodiment.

FIG. 2 is a diagram illustrating a schematic configuration example of a right-foot measurement instrument according to the first embodiment.

FIG. 3 is a diagram illustrating a schematic configuration example of a mobile terminal device according to the first embodiment.

FIG. 4 is a diagram illustrating a schematic configuration example of a server device according to the first embodiment.

FIG. 5 is a diagram illustrating an example of a procedure of a process performed in the first information processing system according to the first embodiment.

FIG. 6 is a diagram illustrating an example of a procedure of a process during running measurement performed in the first information processing system according to the first embodiment.

FIG. 7 is a diagram illustrating an example of a procedure of a process after running measurement performed in the first information processing system according to the first embodiment.

FIG. 8 is a diagram illustrating an example of a procedure of a landing method determination process performed in the first information processing system according to the first embodiment.

FIG. 9 is a diagram illustrating an example of a procedure of a pronation determination process performed in the first information processing system according to the first embodiment.

FIG. 10 is a diagram illustrating a first example of a procedure of a suggested shoes determination process performed in the first information processing system according to the first embodiment.

FIG. 11 is a diagram illustrating a second example of the procedure of the suggested shoes determination process performed in the first information processing system according to the first embodiment.

FIG. 12 is a diagram illustrating a third example of the procedure of the suggested shoes determination process performed in the first information processing system according to the first embodiment.

FIG. 13 is a diagram illustrating an example of preprocessing for the first and second examples of the procedure of the suggested shoes determination process performed in the first information processing system according to the first embodiment.

FIG. 14 is a diagram illustrating an example of preprocessing for the third example of the procedure of the suggested shoes determination process performed in the first information processing system according to the first embodiment.

FIG. 15 is a diagram illustrating an example of a procedure of a suggested shoes filtering process performed in the first information processing system according to the first embodiment.

FIG. 16A is a diagram illustrating an example of a table of correspondence between a landing score, a pronation score, and the like, and suggested shoes according to the first embodiment.

FIG. 16B is a diagram illustrating an example of the table of correspondence between the landing score, the pronation score, and the like, and the suggested shoes according to the first embodiment.

FIG. 16C is a diagram illustrating an example of the table of correspondence between the landing score, the pronation score, and the like, and the suggested shoes according to the first embodiment.

FIG. 17 is a diagram illustrating a schematic configuration example of a second information processing system according to a second embodiment.

### DESCRIPTION OF EXEMPLARY EMBODIMENTS

Embodiments will be described below with reference to the accompanying figures.

A first embodiment will be described.

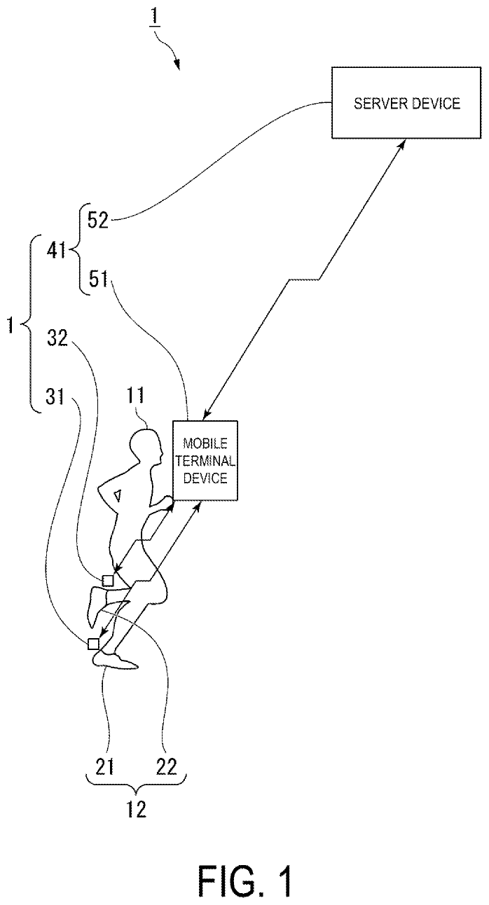

FIG. 1 is a diagram illustrating a schematic configuration example of a first information processing system 1 according to the first embodiment.

The first information processing system 1 includes a right-foot measurement instrument 31, a left-foot measurement instrument 32, and a first information processing device 41.

The first information processing device 41 includes a mobile terminal device 51 and a server device 52.

FIG. 1 illustrates a first user 11 and first shoes 12.

The first shoes 12 include a right-foot shoe 21 and a left-foot shoe 22.

The first user 11 wears the right-foot shoe 21 on the right foot of the first user 11 and the left-foot shoe 22 of the left foot of the first user 11.

The right-foot measurement instrument 31 is attached to the right foot of the first user 11 or the right-foot shoe 21. Note that the right-foot measurement instrument 31 may be included in a sole of the right-foot shoe 21.

The left-foot measurement instrument 32 is attached to the left foot of the first user 11 or the left-foot shoe 22. Note that the left-foot measurement instrument 32 may be included in a sole of the left-foot shoe 22.

The mobile terminal device 51 is carried by the first user 11.

The server device 52 is connected to a network. Note that in the embodiment, illustration of the network is omitted. The network may be a public network such as the Internet or a dedicated network.

The server device 52 may be provided to any location.

In the embodiment, communication is made between the right-foot measurement instrument 31 and the mobile terminal device 51. The communication may be radio communication, for example.

In the embodiment, communication is made between the left-foot measurement instrument 32 and the mobile terminal device 51. The communication may be radio communication, for example.

In the embodiment, communication is made between the mobile terminal device 51 and the server device 52. The communication is made via a base station device and mobile phone network of a mobile phone system to which the mobile terminal device 51 belongs. Note that in the embodiment, illustrations of the base station device and the mobile phone network are omitted.

Here, each of the right-foot measurement instrument 31 and the left-foot measurement instrument 32 may be provided to any location.

For example, when each of the right-foot measurement instrument 31 and the left-foot measurement instrument 32 includes a sensor detecting data about running motion, the sensor is provided, lower than knees of the first user 11, to an ankle of the right foot or an ankle of the left foot, or to the right-foot shoe 21 or the left-foot shoe 22, or to the sole of the right-foot shoe 21 or the sole of the left-foot shoe 22.

Note that although the embodiment describes a case that each of the right-foot measurement instrument 31 and the left-foot measurement instrument 32 is an instrument integrated with the sensor, another example may use a configuration in which each of the right-foot measurement instrument 31 and the left-foot measurement instrument 32 separately includes a sensor and a main body other than the sensor.

The embodiment describes a case that the first user 11 wears the first shoes 12 to run at a road.

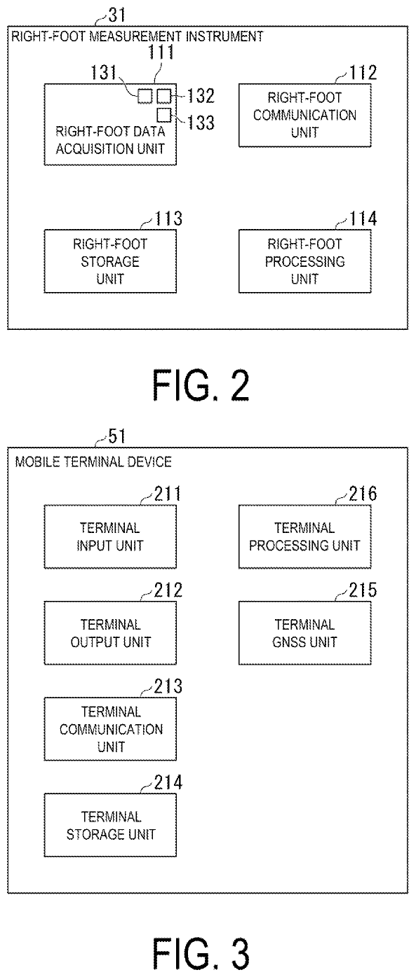

FIG. 2 is a diagram illustrating a schematic configuration example of the right-foot measurement instrument 31 according to the first embodiment.

Note that in the embodiment, a configuration and operation of the left-foot measurement instrument 32 are schematically the same as or similar to a configuration and operation of the right-foot measurement instrument 31. Specifically, a description of the left-foot measurement instrument 32 may be obtained by interpreting the “right-foot” in a description of the right-foot measurement instrument 31 as the “left-foot”.

However, the right-foot measurement instrument 31 and the left-foot measurement instrument 32 are assigned with pieces of identification information different from each other, and are identified to be distinguished by use of these pieces of identification information. When the right-foot measurement instrument 31 and the left-foot measurement instrument 32 need not be distinguished, such identification information need not necessarily be used.

Such identification information may include, for example, information identifying the first shoes 12 including the right-foot measurement instrument 31 and the left-foot measurement instrument 32. In this case, the first shoes 12 worn by the first user 11 can be specified by use of the pieces of identification information.

The first shoes 12 worn by the first user 11 may be automatically identified by the mobile terminal device 51 or the server device 52, based on such identification information.

In another example, a configuration may be used in which the mobile terminal device 51 identifies the first shoes 12 worn by the first user 11, based on an operation performed on the mobile terminal device 51 by the first user 11.

The right-foot measurement instrument 31 includes a right-foot data acquisition unit 111, a right-foot communication unit 112, a right-foot storage unit 113, and a right-foot processing unit 114.

The right-foot data acquisition unit 111 includes a right-foot acceleration sensor 131 that is an acceleration sensor, a right-foot gyroscope sensor 132 that is a gyroscope sensor, and a right-foot Global Navigation Satellite System (GNSS) unit 133.

Note that the acceleration sensor and the gyroscope sensor may be collectively referred to as an inertia sensor.

The right-foot data acquisition unit 111 acquires prescribed data.

In the embodiment, the prescribed data includes data detected by the right-foot acceleration sensor 131, data detected by the right-foot gyroscope sensor 132, and data detected by the right-foot GNSS unit 133.

Note that these piece of detected data may be included in the prescribed data as data detected without change or as data converted from the detected data.

In other words, the right-foot data acquisition unit 111 may deal with these pieces of detected data without change, or may deal with data obtained by converting part or all of these pieces of detected data.

Here, the right-foot acceleration sensor 131 detects data of accelerations of three axes orthogonal to each other.

The right-foot acceleration sensor 131 may detect, for example, data about impact on the right foot of the first user 11 when the right-foot shoe 21 lands. Timing at which the right-foot shoe 21 lands may be, for example, timing at which an acceleration detected by the right-foot acceleration sensor 131 reaches a prescribed value or more.

Note that the right-foot acceleration sensor 131 may detect the data at every prescribed period. In this case, the data when the right-foot shoe 21 lands may be extracted by the server device 52 or the like, based on the data detected by the right-foot acceleration sensor 131.

The right-foot gyroscope sensor 132 detects data of angular velocities about three axes orthogonal to each other.

The right-foot gyroscope sensor 132 may detect, for example, data about the right foot of the first user 11 when the right-foot shoe 21 lands. Timing at which the right-foot shoe 21 lands may be, for example, timing at which an acceleration detected by the right-foot acceleration sensor 131 reaches a prescribed value or more.

Note that the right-foot gyroscope sensor 132 may detect the data at every prescribed period. In this case, the data when the right-foot shoe 21 lands may be extracted by the server device 52 or the like, based on the data detected by the right-foot gyroscope sensor 132.

Note that in a configuration in which such gyroscope sensor data is not used, the right-foot measurement instrument 31 need not necessarily include the right-foot gyroscope sensor 132.

The right-foot GNSS unit 133, which has a function of a GNSS, receives signals transmitted by radio from a GNSS satellite and detects data based on the signals. The data includes, for example, data of a position of the right-foot GNSS unit 133 and data of time.

Note that in a configuration in which such data is not used, the right-foot measurement instrument 31 need not necessarily include the right-foot GNSS unit 133.

Here, in the function of the GNSS, a GNSS reception unit receives an electric wave transmitted from a navigation satellite by using a GNSS antenna and analyzes the receive electric wave by using a GNSS circuit, to acquire GNSS information. The GNSS information includes, for example, positional information including a latitude, a longitude, and an altitude, and information about a time of day and a date. In the example in FIG. 2, the right-foot GNSS unit 133 has the function of the GNSS reception unit.

A Global Positioning System (GPS) may be used as the GNSS, for example.

Note that in the embodiment, illustrations of the GNSS reception unit, the GNSS antenna, and the GNSS circuit are omitted.

The right-foot communication unit 112 has a function to communicate by radio with the mobile terminal device 51.

The right-foot communication unit 112 transmits data acquired by the right-foot data acquisition unit 111 to the mobile terminal device 51.

The right-foot storage unit 113 has a function to store data.

The right-foot storage unit 113 may store, for example, the data acquired by the right-foot data acquisition unit 111, or data obtained based on the former data.

The right-foot storage unit 113 may store, for example, the identification information of the right-foot measurement instrument 31 or the like.

Note that in the right-foot measurement instrument 31, the right-foot storage unit 113 need not be included when the data is not necessarily stored.

The right-foot processing unit 114, which includes a processor such as a Central Processing Unit (CPU), for example, has a function to perform processing.

The processor reads, for example, a program stored in the right-foot storage unit 113 to perform processing in accordance with the program.

Note that in the right-foot measurement instrument 31, the right-foot processing unit 114 need not necessarily be included when processing need not be performed by the processor in the right-foot processing unit 114.

In the embodiment, the right-foot measurement instrument 31 acquires data about the right-foot shoe 21 of the first shoes 12 used by the first user 11 and transmits the acquired data to the mobile terminal device 51.

The left-foot measurement instrument 32 acquires data about the left-foot shoe 22 of the first shoes 12 used by the first user 11 and transmits the acquired data to the mobile terminal device 51. By doing so, the right-foot measurement instrument 31 and the left-foot measurement instrument 32 acquire the data about the first shoes 12 used by the first user 11 and transmit the acquired data to the mobile terminal device 51.

Here, the data about the right-foot shoe 21 may reflect a feature of a running style of the right foot of the first user 11 or the like. The data about the left-foot shoe 22 may reflect a feature of a running style of the left foot of the first user 11 or the like. The data about the first shoes 12 may reflect a feature of a running style of the first user 11 or the like.

FIG. 3 is a diagram illustrating a schematic configuration example of the mobile terminal device 51 according to the first embodiment.

The mobile terminal device 51 is a smartphone in the embodiment, but may be another device. Such another device may include, for example, a mobile phone terminal device, a mobile small computer, or the like.

The mobile terminal device 51 includes a terminal input unit 211, a terminal output unit 212, a terminal communication unit 213, a terminal storage unit 214, a terminal GNSS unit 215, and a terminal processing unit 216.

The terminal input unit 211, which includes an operation unit accepting an operation performed by the first user 11, inputs information corresponding to the operation.

The terminal output unit 212, which includes a screen, displays information on the screen to output the information.

Here, the operation unit included in the terminal input unit 211 and the screen included in the terminal output unit 212 may be configured as a display screen with a touch panel.

The terminal input unit 211 may have a function to input voice or the like. The terminal output unit 212 may have a function to output voice or the like.

The terminal communication unit 213 has a function to communicate by radio with each of the right-foot measurement instrument 31 and the left-foot measurement instrument 32. In the communication, the identification information of each of the right-foot measurement instrument 31 and the left-foot measurement instrument 32 may be included in a communication signal to identify each of the right-foot measurement instrument 31 and the left-foot measurement instrument 32.

Note that the communication between the terminal communication unit 213 and each of the right-foot measurement instrument 31 and the left-foot measurement instrument 32 may include, for example, communication using Bluetooth (registered trademark), communication using Bluetooth Low Energy (registered trademark), communication using Long Term Evolution (LTE), communication using Wi-Fi (registered trademark), Near Field Communication (NFC), and the like.

As a concrete example, the right-foot measurement instrument 31 and the left-foot measurement instrument 32 may have a function of Radio Frequency Identifier (RFID) to perform communication using the RFID between the terminal communication unit 213 and each of the right-foot measurement instrument 31 and the left-foot measurement instrument 32. In the example in FIG. 2, the right-foot communication unit 112 in the right-foot measurement instrument 31 may have the function of RFID, and the same holds for the left-foot measurement instrument 32.

The terminal communication unit 213 has a function to communicate with the server device 52. In the embodiment, the terminal communication unit 213, which has a function to communicate by radio with the base station device of the mobile phone system, communicates with the server device 52 via the base station device and the mobile phone network.

Note that in the embodiment, for convenience of description, the terminal communication unit 213 has a function to communicate with each of the right-foot measurement instrument 31 and the left-foot measurement instrument 32, and a function to communicate with the server device 52, but these functions may be configured as functions of separate processing units.

The terminal storage unit 214 has a function to store data.

The terminal storage unit 214 may store, for example, data received from each of the right-foot measurement instrument 31 and the left-foot measurement instrument 32, data received from the server device 52, or a different piece of data.

The terminal GNSS unit 215, which has the function of the GNSS, receives signals transmitted by radio from the GNSS satellite and detects data based on the signals. The data includes, for example, data of a position of the terminal GNSS unit 215 and data of time.

Note that in a configuration in which such data is not used, the mobile terminal device 51 need not necessarily include the terminal GNSS unit 215.

The terminal processing unit 216, which includes a processor such as a CPU, for example, has a function to perform processing.

The processor reads, for example, a program stored in the terminal storage unit 214 to perform processing in accordance with the program.

In the embodiment, the mobile terminal device 51 relays communication between each of the right-foot measurement instrument 31 and the left-foot measurement instrument 32 and the server device 52.

Specifically, the mobile terminal device 51 receives, by use of the terminal communication unit 213, data transmitted from each of the right-foot measurement instrument 31 and the left-foot measurement instrument 32, and transmits the received data to the server device 52.

The mobile terminal device 51 receives data transmitted from the server device 52 to each of the right-foot measurement instrument 31 and the left-foot measurement instrument 32, and transmits the received data to each of the right-foot measurement instrument 31 and the left-foot measurement instrument 32. Note that a configuration may be used in which data is not transmitted in a direction from the server device 52 to each of the right-foot measurement instrument 31 and the left-foot measurement instrument 32.

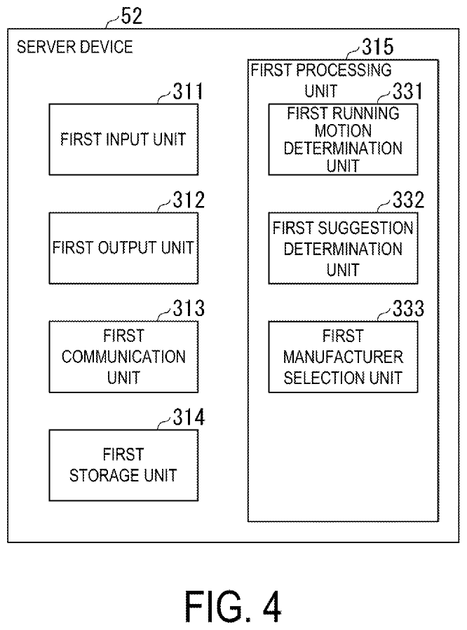

FIG. 4 is a diagram illustrating a schematic configuration example of the server device 52 according to the first embodiment.

The server device 52 is configured using, for example, a computer.

The server device 52 includes a first input unit 311, a first output unit 312, a first communication unit 313, a first storage unit 314, and a first processing unit 315.

The first processing unit 315 includes a first running motion determination unit 331, a first suggestion determination unit 332, and a first manufacturer selection unit 333.

The first input unit 311, which includes an operation unit accepting an operation performed by an administrator or the like of the server device 52, inputs information corresponding to the operation.

The first output unit 312, which includes a screen, displays information on the screen to output the information.

The first input unit 311 may have a function to input voice or the like. The first output unit 312 may have a function to output voice or the like.

The first communication unit 313 has a function to communicate with the mobile terminal device 51.

In the embodiment, the first communication unit 313 communicates with the mobile terminal device 51 via the base station device of the mobile phone system.

The first storage unit 314 has a function to store data.

The first storage unit 314 may store, for example, data received from the mobile terminal device 51, or data obtained based on the former data.

The first processing unit 315, which includes a processor such as a CPU, for example, has a function to perform processing.

The processor reads, for example, a program stored in the first storage unit 314 to perform processing in accordance with the program.

In the embodiment, the server device 52 receives data transmitted from each of the right-foot measurement instrument 31 and the left-foot measurement instrument 32 via the mobile terminal device 51.

In the embodiment, the first processing unit 315 performs both processing using the data received from the right-foot measurement instrument 31 and processing using the data receive from the left-foot measurement instrument 32. In the embodiment these processing operations are the same, and thus, the processing using the data received from the right-foot measurement instrument 31 is described, and a detailed description of the processing using the data received from the left-foot measurement instrument 32 is omitted.

Note that the processing about the right-foot measurement instrument 31 is processing about the right-foot shoe 21, and the processing about the left-foot measurement instrument 32 is processing about the left-foot shoe 22.

Here, features of a running style of a human include a landing method and a pronation.

The landing method includes a toe landing, a flat landing, and a heel landing.

The pronation includes an overpronation, a neutral pronation, and an underpronation. Note that the neutral pronation may be referred to as a natural pronation.

In the embodiment, a landing score that is a score for the landing method and a pronation score that is a score for the pronation are used.

The pronation score includes a score for the overpronation and a score for the underpronation. The pronation score includes a score for the neutral pronation that is not an overpronation or an underpronation.

The landing score includes a score for the toe landing and a score for the heel landing. The landing score includes a score for the flat landing that is not a score for the toe landing or a score for the heel landing.

At least one of the score for the overpronation, the score for the underpronation, the score for the toe landing, and the score for the heel landing has a value in a plurality of stages. The value for each of the plurality of stages may be any value, for example, may be a positive or negative integer value, a positive integer value, or a negative integer value. The values for the plurality of stages may include, for example, a value including not only an integer but also a decimal, or a sign such as A, B, C and so on.

Note that the values for the plurality of stages may be referred to as degrees.

The first processing unit 315 acquires a measurement result including at least a roll angle upon landing and a pitch angle upon landing, based on data received from the right-foot measurement instrument 31 by the first communication unit 313. The measurement result may include information about a running velocity.

The embodiment performs inertial navigation based on a detection result of a six-axis inertia sensor obtained by combining the right-foot acceleration sensor 131 with three axes and the right-foot gyroscope sensor 132 with three axes which are worn by the first user 11 be able to specify a roll angle, pitch angle, and yaw angle expressing a posture of the right-foot shoe 21.

Here, the processing for calculating the roll angle, the pitch angle, and the yaw angle from the data detected by the right-foot acceleration sensor 131 and the right-foot gyroscope sensor 132 may be performed by, for example, the right-foot data acquisition unit 111 in the right-foot measurement instrument 31, the first processing unit 315 in the server device 52, or the terminal processing unit 216 in the mobile terminal device 51.

The processing may be distributively performed by, for example, any two or more of the right-foot measurement instrument 31, the server device 52, and the mobile terminal device 51.

Note that when the processing may be performed by the first processing unit 315 in the server device 52, the processing may be performed by, for example, the first running motion determination unit 331.

The first running motion determination unit 331 performs at least determination of the pronation score by comparison between the roll angle and a first threshold and determination of the landing score by comparison between the pitch angle and a second threshold.

Here, the pronation score determination may be made by, for example, calculating the pronation score using a predefined arithmetic expression, or the like. Note that the arithmetic expression may be able to be updated.

The embodiment describes a case that a score at the time of neutral pronation is 0 point, a score at the time of underpronation is a positive value, and a score at the time of overpronation is a negative value.

As the first threshold for the roll angle, two or more thresholds may be used.

For example, as the first threshold, a first 1-1 threshold for determining the underpronation and a first 1-2 threshold for determining the overpronation may be used.

The first running motion determination unit 331 determines the pronation of the right foot, based on the roll angle of the right-foot shoe 21 upon landing that is at timing of landing.

The first running motion determination unit 331 determines the underpronation when the roll angle upon landing is equal to or greater than a prescribed threshold thresh_under_pronation. In this case, the first running motion determination unit 331 sets the pronation score to a value corresponding to a resultant obtained by subtracting thresh_under_pronation from the roll angle.

In the embodiment, the score for the underpronation has an integer value, for example, a value of +1, +2, or +3.

As a concrete example, the score may start from 0 point with thresh_under_pronation being+5 degrees, and 1 point may be added to the score every time the roll angle increases by 1 degree from thresh_under_pronation.

The first running motion determination unit 331 determines the overpronation when the roll angle upon landing is equal to or less than a prescribed threshold thresh_over_pronation. In this case, the first running motion determination unit 331 sets the pronation score to a value corresponding to a resultant obtained by subtracting thresh_over_pronation from the roll angle.

In the embodiment, the score for the overpronation has an integer value, for example, a value of −1, −2, or −3.

As a concrete example, the score may start from 0 point with thresh_over_pronation being −5 degrees, and 1 point may be added to the score every time the roll angle decreases by 1 degree from thresh_over_pronation.

The first running motion determination unit 331 determines the neutral pronation when the roll angle upon landing does not fall into any of the underpronation and the overpronation. In this case, the first running motion determination unit 331 sets the pronation score to 0 point.

The landing method determination may be made by, for example, calculating the landing score using a predefined arithmetic expression, or the like. Note that the arithmetic expression may be able to be updated.

The embodiment describes a case that a score upon flat landing is 0 point, a score upon heel landing is a positive value, and a score upon toe landing is a negative value.

As the second threshold for the pitch angle, two or more thresholds may be used.

For example, as the second threshold, a second 2-1 threshold for determining the toe landing and a second 2-2 threshold for determining the heel landing may be used.

The first running motion determination unit 331 determines the landing method of the right foot, based on the pitch angle of the right-foot shoe 21 upon landing that is at timing of landing.

The first running motion determination unit 331 determines the heel landing when the pitch angle upon landing is equal to or greater than a prescribed threshold thresh_heel_strike_angle. In this case, the first running motion determination unit 331 sets the landing score to a value corresponding to a resultant obtained by subtracting thresh_heel_strike_angle from the pitch angle.

In the embodiment, the score for the heel landing has an integer value, for example, a value of +1, +2, or +3.

As a concrete example, the score may start from 0 point with thresh_heel_strike_angle being +10 degree, and 1 point may be added to the score every time the pitch angle increases by 1 degree from thresh_heel_strike_angle.

The first running motion determination unit 331 determines the toe landing when the pitch angle upon landing is equal to or less than a prescribed threshold thresh_toe_strike_angle. In this case, the first running motion determination unit 331 sets the landing score to a value corresponding to a resultant obtained by subtracting thresh_toe_strike_angle from the pitch angle.

In the embodiment, the score for the toe landing has an integer value, for example, a value of −1, −2, or −3.

As a concrete example, the score may start from 0 point with thresh_toe_strike_angle being −10 degrees, and 1 point is subtracted from the score every time the pitch angle decreases by 1 degree from thresh_toe_strike_angle.

The first running motion determination unit 331 determines the flat landing when the pitch angle upon landing does not fall into any of the heel landing and the toe landing. In this case, the first running motion determination unit 331 sets the landing score to 0 point.

The first running motion determination unit 331 may determine a running velocity score by comparison between the running velocity and a third threshold.

Here, as the third threshold for the running velocity, two or more thresholds may be used.

For example, as the third threshold, m different thresholds each of which is an integer of 1 or more may be used to make determination to classify the running velocities into m+1 ranges.

In the embodiment, as the running velocity score, a score for elementary, a score for intermediate, and a score for advanced are used.

As a concrete example, the first running motion determination unit 331 may configure the score for elementary for an elementary person having the running velocity of 6 min/km or more, the score for intermediate for an intermediate person having the running velocity of 5 min/km-range, and the score for advanced for an advanced person having the running velocity of 4 min/km or less.

Note that the running velocity score may be referred to as a running level or the like.

As another example, the first running motion determination unit 331 may determine a score calculated using a predefined arithmetic expression as the running velocity score.

As a concrete example, the first running motion determination unit 331 may add +0.1 point to the score, the score being 0 point when the running velocity is 7 min/km, every time the running velocity decreases by 1 second. In this case, the score is 6 points when the running velocity is 6 min/km, as an example.

Here, the first running motion determination unit 331 may determine the running velocity based on a temporal change in positional data when the first user 11 is in a running motion.

The first running motion determination unit 331 may use, as the running velocity, an average velocity for a prescribed period or an average velocity for a prescribed distance, for example.

The first running motion determination unit 331 may determine whether or not the first user 11 is in a state of running motion.

For example, the first running motion determination unit 331 may determine that the first user 11 starts running, when an acceleration of the first user 11 reaches a prescribed value A1 or more.

For example, the first running motion determination unit 331 may determine that the first user 11 stops running, when the acceleration of the first user 11 reaches a prescribed value A2 or more in a direction opposite to a forward running direction. For example, the first running motion determination unit 331 may determine that the first user 11 is running, when the velocity of the first user 11 is a prescribed value A3 or more, or that the first user 11 is not running when the velocity of the first user 11 is less than the prescribed value A3.

As another example, a configuration may be used in which the first user 11 at starting running performs a prescribed operation for starting running with respect to any of the right-foot measurement instrument 31, the left-foot measurement instrument 32, or the mobile terminal device 51. In this case, information corresponding to the operation may be transmitted from the instrument or device accepting the operation to the server device 52 to cause the first running motion determination unit 331 to determine that the first user 11 starts running.

Similarly, a configuration may be used in which the first user 11 at stopping running performs a prescribed operation for stopping running with respect to any of the right-foot measurement instrument 31, the left-foot measurement instrument 32, or the mobile terminal device 51. In this case, information corresponding to the operation may be transmitted from the instrument or device accepting the operation to the server device 52 to cause the first running motion determination unit 331 to determine that the first user 11 stops running.

The first running motion determination unit 331 may determine another piece of information as the feature of the running style.

For example, the first running motion determination unit 331 may make a determination for identifying stride frequency running or stride length running.

As a concrete example, the first running motion determination unit 331 may determine, based on the number of steps per prescribed time period such as one minute, the stride frequency running in a case of 180 step/min, or the stride length running in other cases.

The first suggestion determination unit determines the suggested shoes, based on the pronation score and the landing score.

The first suggestion determination unit may determine the suggested shoes, based on, for example, the pronation score of the pronation score and the landing score in some cases.

The first suggestion determination unit may determine the suggested shoes, based on, for example, the landing score of the pronation score and the landing score in some cases.

The first suggestion determination unit may use, as a condition for determining the suggested shoes, information other than the pronation score and the landing score.

For example, the first suggestion determination unit may determine the suggested shoes, based on the pronation score, the landing score, and the running velocity score.

For example, one pair of shoes may be determined as the suggested shoes, or two or more pairs of shoes may be determined as the suggested shoes in some cases.

The first suggestion determination unit may determine, for example, one or more pairs of suggested shoes from among a plurality of candidates for shoes prepared in advance.

Note that the suggested shoes determined by the first suggestion determination unit need not necessarily be a final suggested shoes but may be made to be the candidates for the suggested shoes, and further the shoes suggested may be selected from the candidates, for example.

The first manufacturer selection unit 333 selects a manufacturer of the shoes.

The first suggestion determination unit 332 may select the suggested shoes from among shoes of the manufacturer selected by the first manufacturer selection unit 333.

Note that in the present disclosure, the first manufacturer selection unit 333 is not necessarily an indispensable component.

Here, the first manufacturer selection unit 333 may be configured to select a manufacturer manually by the first user 11, configured to select a manufacturer based on a predefined rule, or configured to select a predefined manufacturer.

The number of selected manufacturers may be any number of 1 or greater.

As a concrete example, the first manufacturer selection unit 333 may be configured to accept a selection of the manufacturer preferred by the first user 11 responding to an operation performed by the first user 11 via a user interface.

The user interface may be a user interface of the mobile terminal device 51, for example. In this case, the mobile terminal device 51 transmits information specifying the manufacturer accepted from the first user 11 to the server device 52, and thereby, the first manufacturer selection unit 333 in the server device 52 acquires the information.

Note that although the embodiment describes the first manufacturer selection unit 333 and the first suggestion determination unit 332 as separate processing units, the first suggestion determination unit 332 may be configured to include the function of the first manufacturer selection unit 333, as another example.

Here, the embodiment describes the case that each of the right-foot measurement instrument 31, the left-foot measurement instrument 32, and the mobile terminal device 51 has the function of the GNSS, and, for example, the functions of any one or two, or all of these three GNSSs may be used. When the functions of two or more of GNSSs are used, for example, an average of acquisition information of two or more GNSSs may be used.

Note that the function of the GNSS not used may be not be provided. When the function of the GNSS is used, the function of at least one GNSS may be provided.

for example, when the function of the GNSS of the mobile terminal device 51 is used, information based on a signal acquired by the function of the GNSS is included in information transmitted from the mobile terminal device 51 to the server device 52.

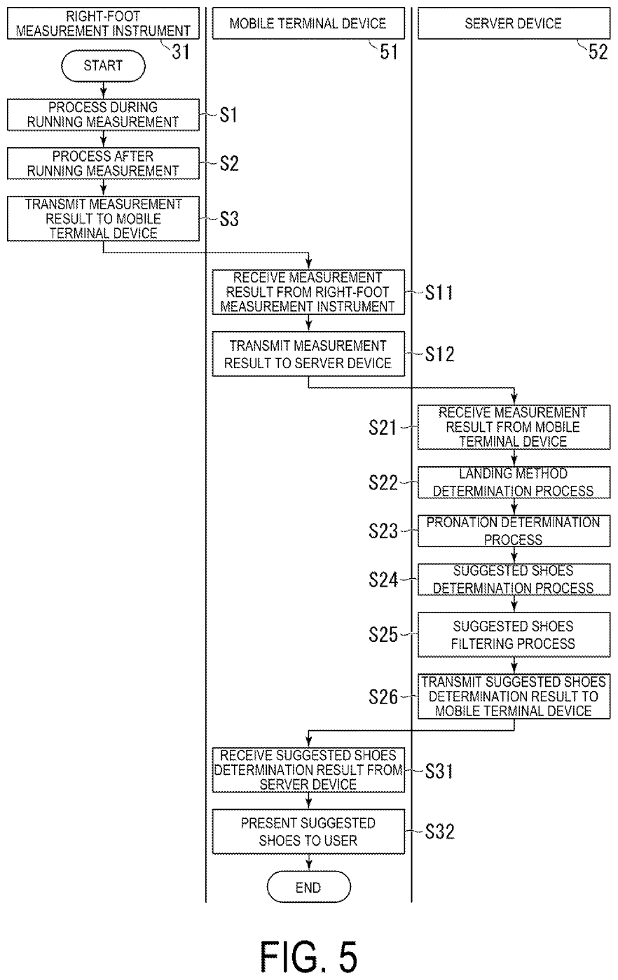

FIG. 5 is a diagram illustrating an example of a procedure of a process performed in the first information processing system 1 according to the first embodiment.

In the embodiment, the right-foot measurement instrument 31 and the left-foot measurement instrument 32 perform similar processes, and thus, the process performed by the right-foot measurement instrument 31 will be described.

The first user 11 starts running in a state of wearing the first shoes 12, and the right-foot measurement instrument 31 and the left-foot measurement instrument 32.

In processing in step S1, the right-foot measurement instrument 31 starts and performs a process during running measurement at the starting of running. Then, the right-foot measurement instrument 31 goes to processing in step S2.

The embodiment measures data in a running motion by use of the right-foot measurement instrument 31 and the left-foot measurement instrument 32 in the state where the first user 11 wears the right-foot measurement instrument 31 and the left-foot measurement instrument 32.

Note that timing at the starting of running may be, as described above, timing at which the first running motion determination unit 331 determines that the first user 11 starts running, for example, or timing at which the first user 11 performs the prescribed operation for starting running with respect to any of the right-foot measurement instrument 31, the left-foot measurement instrument 32, or the mobile terminal device 51, for example.

In the processing in step S2, the right-foot measurement instrument 31 performs a process after running measurement at a prescribed timing after the end of running. Then, the right-foot measurement instrument 31 goes to processing in step S3.

Here, the prescribed timing may be, for example, timing immediately after the end of running.

Note that the timing after the end of running may be, as described above, timing at which the first running motion determination unit 331 determines that the first user 11 stops running, for example, or timing at which the first user 11 performs the prescribed operation for stopping running with respect to any of the right-foot measurement instrument 31, the left-foot measurement instrument 32, or the mobile terminal device 51, for example.

In the processing in step S3, the right-foot measurement instrument 31 transmits the measurement result to the mobile terminal device 51.

Then, the first information processing system 1 goes to processing in step S11.

In the processing in step S11, the mobile terminal device 51 receives the measurement result transmitted from the right-foot measurement instrument 31. Then, the mobile terminal device 51 goes to processing in step S12.

In the processing in step S12, the mobile terminal device 51 transmits the received measurement results to the server device 52.

Then, the first information processing system 1 goes to processing in step S21.

In the processing in step S21, the server device 52 receives the measurement result transmitted from the mobile terminal device 51. Then, the server device 52 goes to processing in step S22.

In the processing in step S22, the server device 52 performs the landing method determination process based on the received measurement results. Then, the server device 52 goes to processing in step S23.

In the processing in step S23, the server device 52 performs the pronation determination process based on the received measurement results. Then, the server device 52 goes to processing in step S24.

Note that the server device 52 may perform the landing method determination process in step S22 and the pronation determination process in step S23 in an arbitrary order or in parallel.

In the processing in step S24, the server device 52 determines the suggested shoes, based on at least the score for the landing method and the score for the pronation. Then, the server device 52 goes to processing in step S25.

Here, the server device 52 may reference another piece of information as the condition for determining the suggested shoes.

In the processing in step S25, the server device 52 performs a filtering process on the suggested shoes. Then, the server device 52 goes to processing in step S26.

Here, the filtering process may include processing about the manufacturer, for example.

Note that as the filtering process, processing about other than the manufacturer may be performed, and for example, processing about one or more of the running level, a price, a preference of the first user 11, or the like may be performed.

In the processing in step S26, the server device 52 transmits a suggested shoes determination result to the mobile terminal device 51.

Then, the first information processing system 1 goes to processing in step S31.

In the processing in step S31, the mobile terminal device 51 receives the suggested shoes determination result transmitted from the server device 52. Then, the mobile terminal device 51 goes to processing in step S32.

In the processing in step S32, the mobile terminal device 51 presents the received suggested shoes determination result to the first user 11.

Then, the first information processing system 1 ends the processing of this flow.

Here, timing at which the mobile terminal device 51 presents the suggested shoes to the first user 11 may be any timing.

For example, a configuration may be used in which the suggested shoes is presented when the first user 11 performs a prescribed operation on the mobile terminal device 51. The operation and presentation may be performed, for example, through a prescribed application in the mobile terminal device 51.

For example, a configuration may be used in which the suggested shoes is presented when the mobile terminal device 51 makes a notification of information about presentable suggested shoes and the first user 11 performs the prescribed operation in response to the notification. The operation and presentation may be performed, for example, through a prescribed application in the mobile terminal device 51. The notification may be referred to as an announcement or the like.

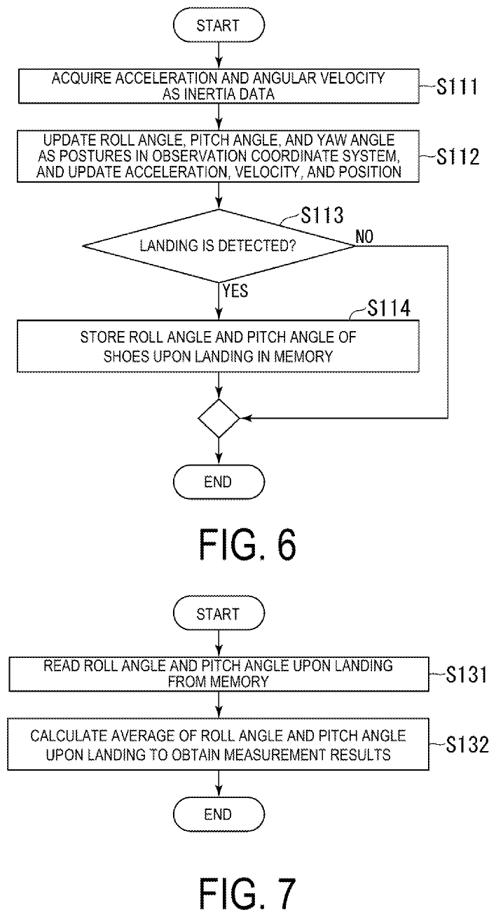

FIG. 6 is a diagram illustrating an example of a procedure of the process during running measurement performed in the first information processing system 1 according to the first embodiment. This example is an example of the processing in step S1 illustrated in FIG. 5.

In processing in step S111, the right-foot measurement instrument 31 acquires, by use of the right-foot data acquisition unit 111, the data of accelerations and the data of angular velocities as the inertia data. Then, the right-foot measurement instrument 31 goes to processing in step S112.

In the processing in step S112, the right-foot measurement instrument 31 updates the roll angle, the pitch angle, and the yaw angle as postures in an observation coordinate system, and updates the acceleration, the velocity, and the position, based on the data acquired by the right-foot data acquisition unit 111. Then, the right-foot measurement instrument 31 goes to processing in step S113.

Here, although this example describes the case that the right-foot measurement instrument 31 performs calculation or the like of the roll angle, the pitch angle, the yaw angle, the acceleration, the velocity, and the position from the detected data, such calculation or the like may be performed by the mobile terminal device 51 or the server device 52 as another example.

In the processing in step S113, the right-foot measurement instrument 31 determines whether or not landing is detected by the right-foot data acquisition unit 111.

As a result of the determination, when the right-foot measurement instrument 31 determines that landing is detected by the right-foot data acquisition unit 111, step S113 results in YES and the right-foot measurement instrument 31 goes to processing in step S114.

On the other hand, as a result of the determination, when the right-foot measurement instrument 31 determines that landing is not detected by the right-foot data acquisition unit 111, step S113 results in NO and the processing of this flow ends.

In the processing in step S114, the right-foot measurement instrument 31 stores, by use of the right-foot data acquisition unit 111. the roll angle and pitch angle of the right-foot shoe 21 upon landing in a memory. As the memory, for example, a memory of the right-foot storage unit 113 may be used. Then, the right-foot measurement instrument 31 ends the processing of this flow.

In the embodiment, the right-foot measurement instrument 31 repeatedly performs the processing of this flow during the running measurement, for example, every certain period of time.

FIG. 7 is a diagram illustrating an example of a procedure of the process after running measurement performed in the first information processing system 1 according to the first embodiment. This example is an example of the processing in step S2 illustrated in FIG. 5.

In processing in step S131, the right-foot measurement instrument 31 reads, by use of the right-foot data acquisition unit 111, the roll angle and pitch angle upon landing from the memory. Then, the right-foot measurement instrument 31 goes to processing in step S132.

In the processing in step S132, the right-foot measurement instrument 31 calculates, by use of the right-foot data acquisition unit 111, averages of the roll angles and pitch angles upon landing in the memory to acquire the measurement result as the averages.

Then, the right-foot measurement instrument 31 ends the processing of this flow.

Here, although this example describes the case that the right-foot measurement instrument 31 calculates the averages of the roll angles and the pitch angles upon landing, such calculation may be performed by the mobile terminal device 51 or the server device 52 as another example.

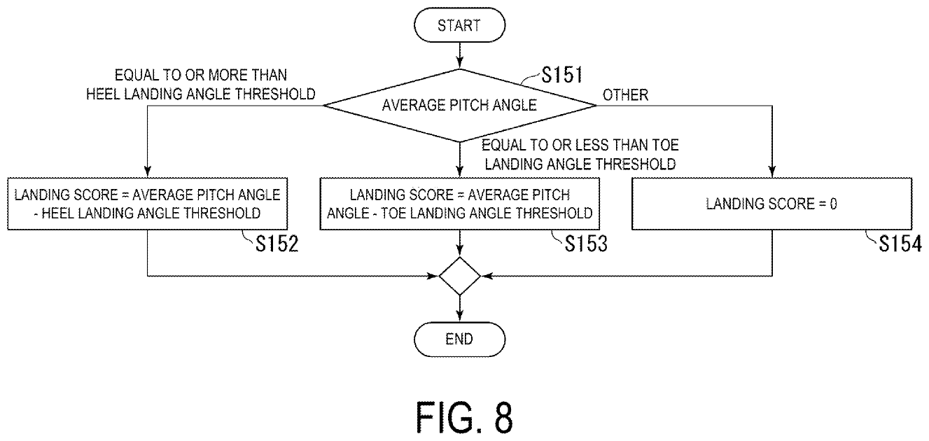

FIG. 8 is a diagram illustrating an example of a procedure of the landing method determination process performed in the first information processing system 1 according to the first embodiment. This example is an example of the processing in step S22 illustrated in FIG. 5.

In processing in step S151, the server device 52 determines, by use of the first running motion determination unit 331, whether or not an average pitch angle that is the average of the pitch angles upon landing is equal to or greater than a threshold of a heel landing angle, whether or not the average pitch angle is equal to or less than a threshold of a toe landing angle, or whether or not the average pitch angle has other value.

Here, in the embodiment, the threshold of the heel landing angle corresponds to thresh_heel_strike_angle, and the threshold of the toe landing angle corresponds to thresh_toe_strike_angle.

As a result of the determination, when the server device 52 determines, by use of the first running motion determination unit 331, that the average pitch angle is equal to or greater than the threshold of the heel landing angle, the server device 52 goes to processing in step S152. As a result of the determination, when the server device 52 determines, by use of the first running motion determination unit 331, that the average pitch angle is equal to or less than the threshold of the toe landing angle, the server device 52 goes to processing in step S153. As a result of the determination, when the server device 52 determines, by use of the first running motion determination unit 331, that the average pitch angle has other value, the server device 52 goes to processing in step S154.

In the processing in step S152, the server device 52 sets as the landing score, by use of the first running motion determination unit 331, a value corresponding to a resultant obtained by subtracting the threshold of the heel landing angle from the average pitch angle.

Then, the server device 52 ends the processing of this flow.

In the processing in step S153, the server device 52 sets as the landing score, by use of the first running motion determination unit 331, a value corresponding to a resultant obtained by subtracting the threshold of the toe landing angle from the average pitch angle.

Then, the server device 52 ends the processing of this flow.

In the processing in step S154, the server device 52 sets, by use of the first running motion determination unit 331, the landing score to 0.

Then, the server device 52 ends the processing of this flow.

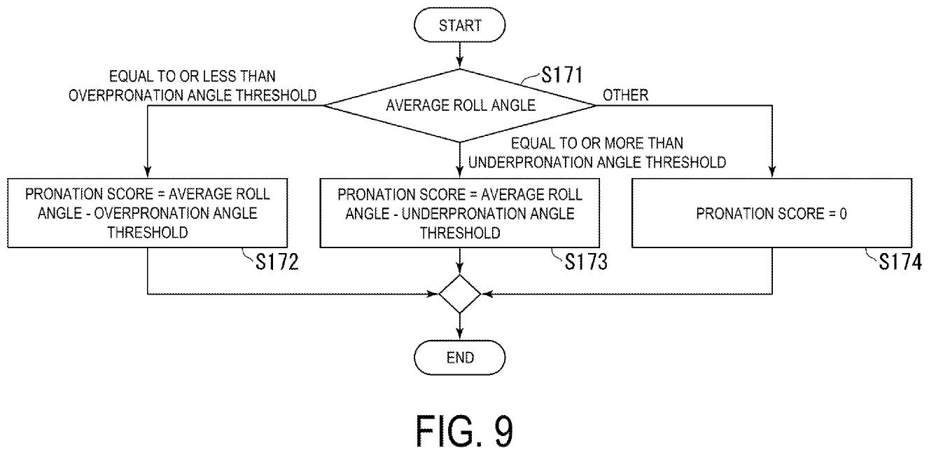

FIG. 9 is a diagram illustrating an example of a procedure of the pronation determination process performed in the first information processing system 1 according to the first embodiment. This example is an example of the processing in step S23 illustrated in FIG. 5.

In processing in step S171, the server device 52 determines, by use of the first running motion determination unit 331, whether or not an average roll angle that is the average of the roll angles upon landing is equal to or less than a threshold of an overpronation angle, whether or not the average roll angle is equal to or greater than a threshold of an underpronation angle, or whether or not the average roll angle has other value.

Here, in the embodiment, the threshold of the overpronation angle corresponds to thresh_over_pronation, and the threshold of the underpronation angle corresponds to thresh_under_pronation.

As a result of the determination, when the server device 52 determines, by use of the first running motion determination unit 331, that the average roll angle is equal to or less than the threshold of the overpronation angle, the server device 52 goes to processing in step S172.

As a result of the determination, when the server device 52 determines, by use of the first running motion determination unit 331, that the average roll angle is equal to or greater than the threshold of the underpronation angle, the server device 52 goes to processing in step S173.

As a result of the determination, when the server device 52 determines, by use of the first running motion determination unit 331, the average roll angle has other value, the server device 52 goes to processing in step S174.

In the processing in step S172, the server device 52 sets as the pronation score, by use of the first running motion determination unit 331, a value corresponding to a resultant obtained by subtracting the threshold of the overpronation angle from the average roll angle.

Then, the server device 52 ends the processing of this flow.

In the processing in step S173, the server device 52 sets as the pronation score, by use of the first running motion determination unit 331, a value corresponding to a resultant obtained by subtracting the threshold of the underpronation angle from the average roll angle.

Then, the server device 52 ends the processing of this flow.

In the processing in step S174, the server device 52 sets, by use of the first running motion determination unit 331, the pronation score to 0.

Then, the server device 52 ends the processing of this flow.

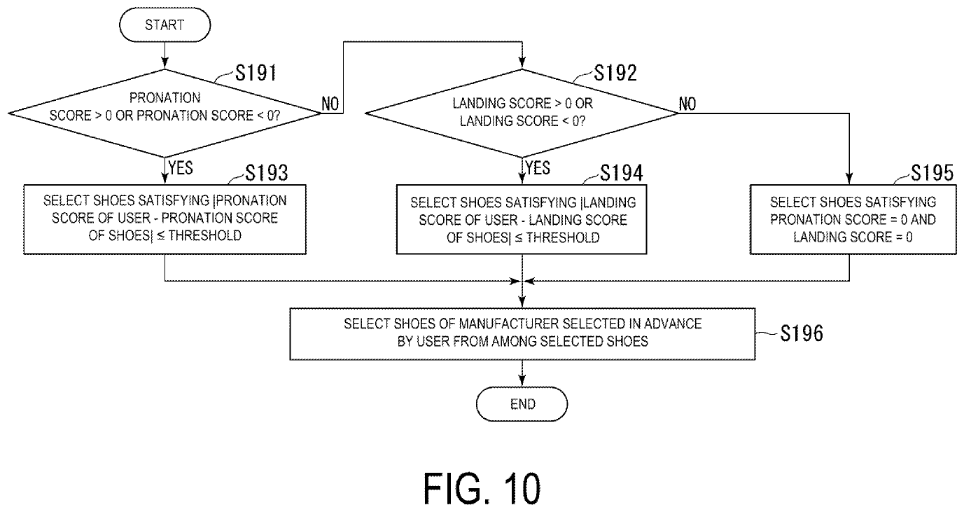

FIG. 10 is a diagram illustrating a first example of a procedure of a suggested shoes determination process performed in the first information processing system 1 according to the first embodiment. This example is an example of the processing in step S24 illustrated in FIG. 5.

In processing in step S191, the server device 52 determines, by use of the first suggestion determination unit 332, whether the pronation score is greater than 0 or the pronation score is less than 0.

As a result of the determination, when the server device 52 determines, by use of the first suggestion determination unit 332, that the pronation score is greater than 0 or the pronation score is less than 0, step S191 results in YES and the server device 52 goes to processing in step S193.

On the other hand, as a result of the determination, when the server device 52 determines, by use of the first suggestion determination unit 332, that the pronation score is 0, step S191 results in NO and the server device 52 goes to processing in step S192.

In the processing in step S192, the server device 52 determines, by use of the first suggestion determination unit 332, whether the landing score is greater than 0 or the landing score is less than 0. As a result of the determination, when the server device 52 determines, by use of the first suggestion determination unit 332, that the landing score is greater than 0 or the landing score is less than 0, step S192 results in YES and the server device 52 goes to processing in step S194.

On the other hand, as a result of the determination, when the server device 52 determines, by use of the first suggestion determination unit 332, that the landing score is 0, step S192 results in NO and the server device 52 goes to processing in step S195.

In the processing in step S193, the server device 52 sequentially finds, by use of the first suggestion determination unit 332, a resultant obtained by subtracting each of the pronation scores of the plurality of candidates for the shoes prepared in advance described above from the pronation score of the first user 11 and taking an absolute value of the subtraction resultant, and selects shoes with the resultant being equal to or less than the prescribed threshold from among the plurality of candidates for the shoes. Then, the server device 52 goes to processing in step S196.

Here, the pronation scores of the plurality of candidates for the shoes are set in advance.

In the processing in step S194, the server device 52 sequentially finds, by use of the first suggestion determination unit 332, a resultant obtained by subtracting each of the landing scores of the plurality of candidates for the shoes prepared in advance described above from the landing score of the first user 11 and taking an absolute value of the subtraction resultant, and selects shoes with the resultant being equal to or less than the prescribed threshold from among the plurality of candidates for the shoes. Then, the server device 52 goes to processing in step S196.

Here, the landing scores of the plurality of candidates for the shoes are set in advance.

In the processing in step S195, the server device 52 selects, by use of the first suggestion determination unit 332, the shoes with the pronation score of 0 and the landing score of 0. Then, the server device 52 goes to processing in step S196.

In the processing in step S196, the server device 52 selects, by use of the first suggestion determination unit 332, shoes of the manufacturer selected in advance by the first user 11 from among the selected shoes.

Then, the server device 52 ends the processing of this flow.

Note that although this example describes the case that the manufacturer is selected in advance by the first user 11, the manufacturer may be configured to be automatically selected as another example. The manufacturer need not necessarily be selected.

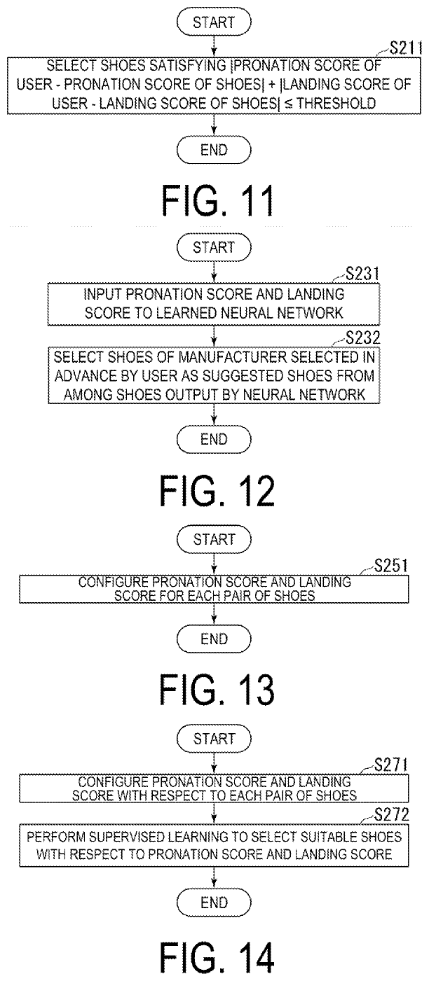

FIG. 11 is a diagram illustrating a second example of the procedure of the suggested shoes determination process performed in the first information processing system 1 according to the first embodiment. This example is an example of the processing in step S24 illustrated in FIG. 5.

In processing in step S211, the server device 52 sequentially finds, by use of the first suggestion determination unit 332, a resultant obtained by subtracting each of the pronation scores of the plurality of candidates for the shoes prepared in advance described above from the pronation score of the first user 11 and taking an absolute value of the subtraction resultant. The server device 52 sequentially finds, by use of the first suggestion determination unit 332, a resultant obtained by subtracting each of the landing scores of the plurality of candidates for the shoes prepared in advance described above from the landing score of the first user 11 and taking an absolute value of the subtraction resultant. Then, the server device 52 adds, by use of the first suggestion determination unit 332, these two resultants to obtain a resultant. The server device 52 selects, by use of the first suggestion determination unit 332, shoes with the addition resultant being equal to or less than a prescribed threshold as the suggested shoes from among the plurality of candidates for the shoes.

Then, the server device 52 ends the processing of this flow.

FIG. 12 is a diagram illustrating a third example of the procedure of the suggested shoes determination process performed in the first information processing system 1 according to the first embodiment. This example is an example of the processing in step S24 illustrated in FIG. 5.

This example is an example using a machine learning result by use of a neural network. In this example, the first suggestion determination unit 332 has a function to perform arithmetic operations for determining an optimal solution based on the machine learning result. The determination may be a presumptive determination.

In processing in step S231, the server device 52 inputs, by use of the first suggestion determination unit 332, the pronation score and the landing score to the learned neural network. Then, the server device 52 goes to processing in step S232.

In the processing in step S232, the server device 52 selects, by use of the first suggestion determination unit 332, shoes of the manufacturer selected in advance by the first user 11 as the suggested shoes from among the shoes output by the neural network.

Then, the server device 52 ends the processing of this flow.

FIG. 13 is a diagram illustrating an example of preprocessing for the first and second examples of the procedure of the suggested shoes determination process performed in the first information processing system 1 according to the first embodiment. The preprocessing is performed in advance, for example, before the processing flow illustrated in FIG. 5 is performed.

In processing in step S251, the server device 52 configures, by use of the first suggestion determination unit 332, the pronation score and the landing score with respect to each pair of shoes.

Then, the server device 52 ends the processing of this flow.

Note that an example of each pair of shoes is the plurality of candidates for the shoes prepared in advance described above.

Here, the pronation score and landing score for each pair of shoes may be configured based on information supplied from a manufacturer of each pair of shoes, or may be manually configured by an administrator or the like via the first input unit 311, for example.

FIG. 14 is a diagram illustrating an example of preprocessing for the third example of the procedure of the suggested shoes determination process performed in the first information processing system 1 according to the first embodiment. The preprocessing is performed in advance, for example, before the processing flow illustrated in FIG. 5 is performed.

In processing in step S271, the server device 52 configures, by use of the first suggestion determination unit 332, the pronation score and the landing score with respect to each pair of shoes. Then, the server device 52 goes to processing in step S272.

In the processing in step S272, the server device 52 performs, by use of the first suggestion determination unit 332, supervised learning to select suitable with respect to the pronation score and the landing score. In the learning, the pronation score and landing score configured for each pair of shoes are used.

Then, the server device 52 ends the processing of this flow.

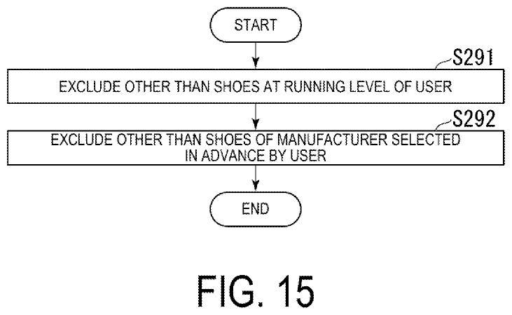

FIG. 15 is a diagram illustrating an example of a procedure of the suggested shoes filtering process performed in the first information processing system 1 according to the first embodiment. This example is an example of the processing in step S25 illustrated in FIG. 5.

In processing in step S291, the server device 52 excludes, by use of the first suggestion determination unit 332, other than the shoes at the running level of the first user 11 from among the suggested shoes. Then, the server device 52 goes to processing in step S292.

In the processing in step S292, the server device 52 excludes, by use of the first suggestion determination unit 332, other than the shoes of the manufacturer selected in advance by the first user 11 from among the suggested shoes.

Then, the server device 52 ends the processing of this flow.

Here, a configuration may be used in which one or both of the exclusion processing in step S291 and the exclusion processing in step S292 are not performed.

Note that although the example in FIG. 15 illustrates the exclusion processing for the manufacturer in step S292, this exclusion processing is similar to the exclusion processing in step S196 illustrated in FIG. 10 and the exclusion processing in step S232 illustrated in FIG. 12, and when the example in FIG. 10 is combined with the example in FIG. 15, any one of the exclusion processing need not necessarily be performed. Similarly, when the example in FIG. 12 is combined with the example in FIG. 15, any one of the exclusion processing need not necessarily be performed. None of the exclusion processing may be performed.

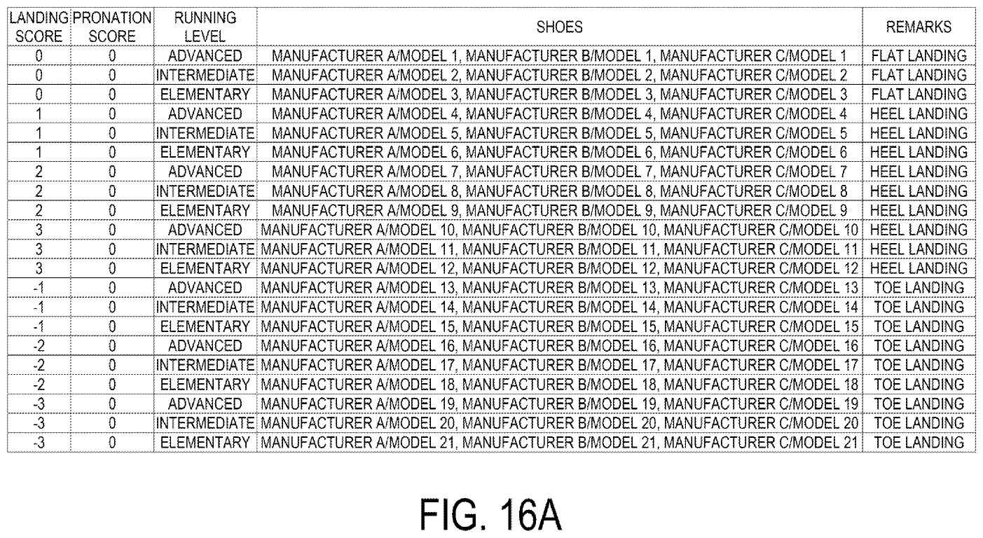

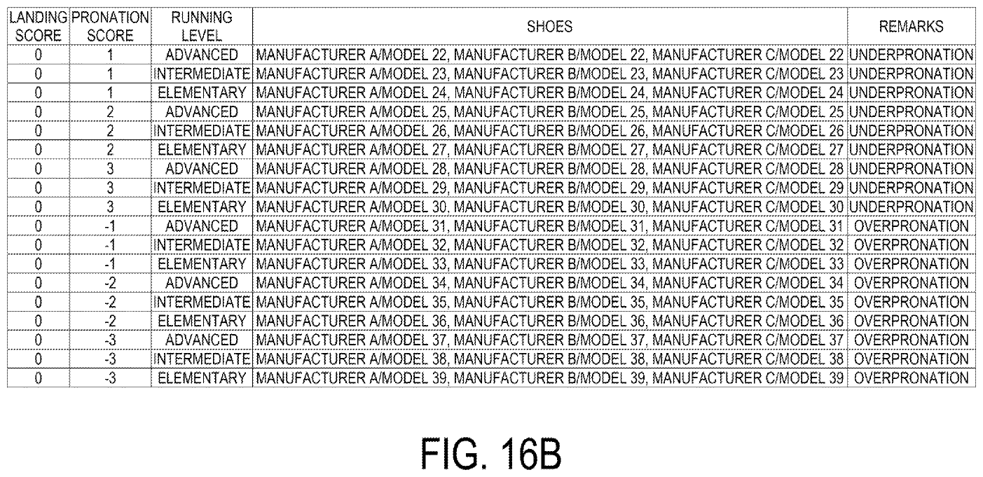

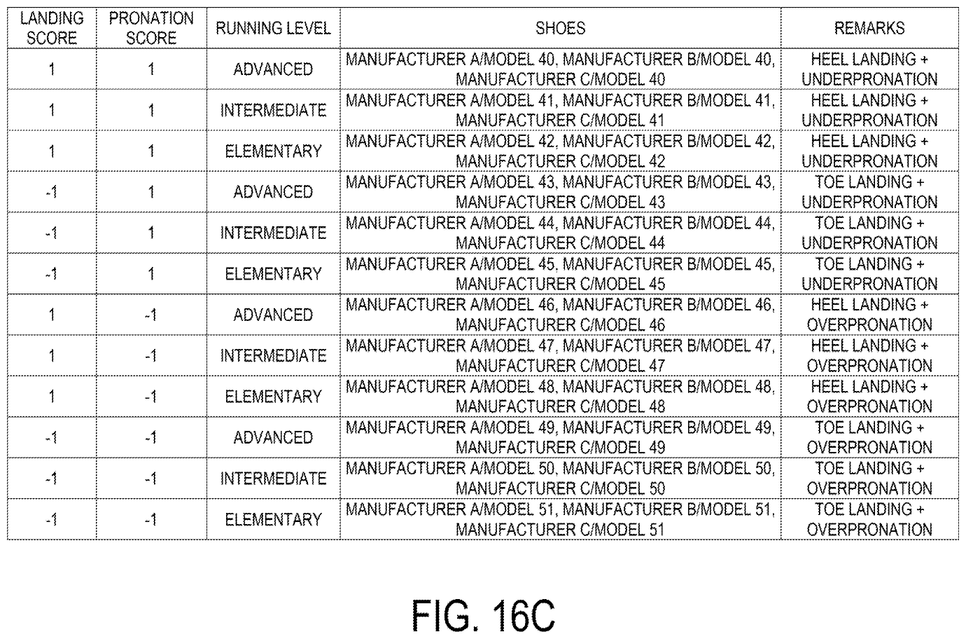

Each of FIGS. 16A, 16B, and 16C is a diagram illustrating an example of a table of correspondence between the landing score, the pronation score, and the like, and the suggested shoes according to the first embodiment. In these correspondence tables, the landing score, the pronation score, the running level, the shoes to be the suggested shoes, and remark information are associated with each other.

In the embodiment, these correspondence tables are stored in the first storage unit 314 in the server device 52.

The landing score has an integer value from −3 to +3.

The pronation score has an integer value from −3 to +3.

The running level is expressed by any of elementary, intermediate, and advanced.

The shoes are specified by a manufacturer and a mode. Note that the shoes may be also specified by a manufacturer, for example.

The remark specifies the toe landing, the heel landing, the flat landing, the overpronation, or the underpronation.

In these correspondence tables, as for a manufacturer of each pair of shoes, information still not selected is stored. In other words, shoes further suggested based on the manufacturer may be selected from a suggest table specified by these correspondence tables.

As another example, a correspondence table storing information about suggested shoes for which a manufacturer is also selected may be used.

In this example, the server device 52 determines, by use of the first suggestion determination unit 332, the shoes suitable to the first user 11, based on the feature of the running style and running level of the first user 11.

In general, concerning the landing method, features of shoes suitable to a person of the heel landing include having a cushioning property of the heel and having a separate sole.

Features of shoes suitable to a person of the flat landing include having a cushioning property and a resilience property for a metatarsal region, and having a flat sole.

Features of shoes suitable to a person of the toe landing include having a cushioning property and a resilience property for a forefoot region, and having a separate sole.

In general, concerning the pronation, features of shoes suitable to a person of the overpronation includes using a firm material for an inside of an outsole.

Features of shoes suitable to a person of the neutral pronation depend on the running level of the person.

Features of shoes suitable to a person of the underpronation includes having a high cushioning property.

In general, concerning the running level, features of shoes suitable to a person of the elementary include having a high cushioning property and having a low resilience property.

Features of shoes suitable to a person of the intermediate include having a high cushioning property and having a resilience property.

Features of shoes suitable to a person of the advanced include having a low cushioning property and having a resilience property.

Note that although the embodiment describes the case that these correspondence tables include information about the shoes of a plurality of different manufacturers, the correspondence table may be separately prepared for the shoes for each manufacturer as another example.

As described above, the first information processing system 1 according to the embodiment can suggest optimal shoes depending on the running style of the first user 11, based on a pattern of the landing such as the toe landing or the heel landing, and a pattern of the pronation expressing a turning degree of the right or left foot.

Therefore, the first information processing system 1 according to the embodiment can determine accurate suggested shoes, and thus, can suggest the shoes suitable to the first user 11, for example, without constraints of place and time.

The first user 11 can receive an introduction to the suggested shoes by running at any place even not a place provided with motion capture using a camera such as at a tread mill.

The first user 11 can also receive an introduction to the suggested shoes by running at any time.

The first user 11 can improve a running ability of the first user 11 by running with wearing the suggested shoes without necessarily improving a physical strength, a muscle strength, and a technique.

The first user 11 can find shoes suitable to the first user 11 without the running of the first user 11 being seen by a specialist.

Note that the first information processing system 1 according to the embodiment may perform, for example, presenting a coupon for the suggested shoes to the first user 11 via the mobile terminal device 51.

When the first user 11 actually uses the coupon to buy shoes, a manufacturer of the shoes may cover a discount due to the coupon for the shop, or an incentive may be given to the administrator or the like of the server device 52.

The first information processing system 1 may perform changing the condition for determining the suggested shoes depending on the incentive from each manufacturer.

The first information processing system 1 may add a feedback evaluation about shoes by another user to determine the suggested shoes for the first user 11. For example, the first information processing system 1 may preferentially suggest, to the first user 11, shoes of which an evaluation by another user is high.

Here, a decision method of an evaluation value of shoes may be any method.

For example, a configuration may be used in which the first user 11 owning the first shoes 12 gives scores of five stages or the like to the first shoes 12.

For example, a configuration may be used in which the evaluation value of the first shoes 12 is given depending on the running style of the first user 11 wearing the first shoes 12.

For example, a configuration may be used in which the evaluation value of shoes is given based on a change in an index before and after the first user 11 exchanges the shoes. The index may include the landing method, the pronation, and the like. The evaluation value may be an evaluation value based on a changed in the running motion after the first user 11 wears the suggested shoes.

The first information processing system 1 can accumulate such an evaluation value and utilize the accumulated evaluation values to select more accurate suggested shoes, for example.

The first information processing system 1 may perform presenting the suggested shoes, for example, for each price range.

The presenting for each price range like this may be performed, for example, at all times independently of a will of the first user 11, or based on an operation of the first user 11. For example, the first information processing system 1 may perform selecting a preferred price range by the first user 11 in advance, and presenting the suggested shoes matching the selected price range to the first user 11.

Here, although the first information processing system 1 according to the embodiment describes the case that the suggested shoes are presented, presenting not only the shoes but also auxiliaries of the shoes such as a sole may be performed, for example.

A second embodiment will be described.

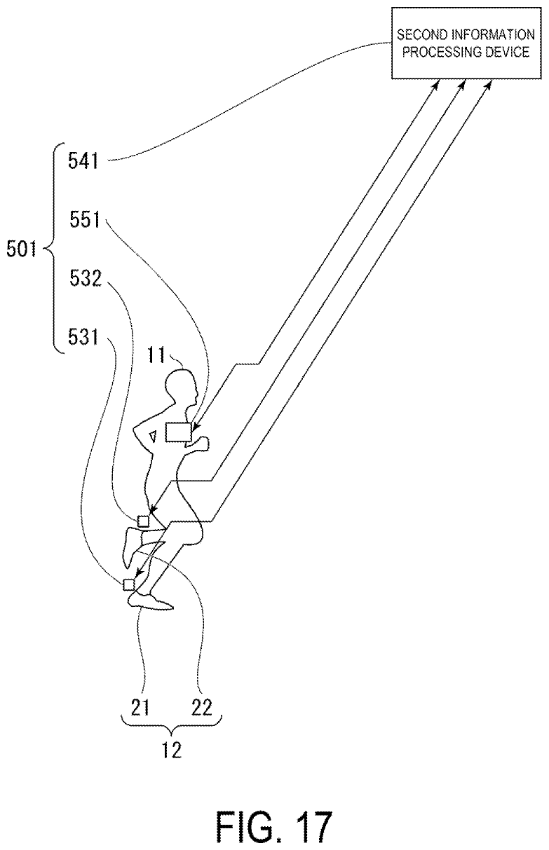

FIG. 17 is a diagram illustrating a schematic configuration example of a second information processing system 501 according to the second embodiment.

Note that, for convenience of description, components similar to the components illustrated in FIG. 1 are denoted by the same reference signs, and detailed descriptions thereof are omitted.

The second information processing system 501 includes a second right-foot measurement instrument 531, a second left-foot measurement instrument 532, a second information processing device 541, and a GNSS unit 551.

FIG. 17 illustrates a first user 11 and a first shoes 12.

The first shoes 12 includes a right-foot shoe 21 and a left-foot shoe 22.

The first user 11 wears the right-foot shoe 21 on a right foot of the first user 11 and the left-foot shoe 22 of a left foot of the first user 11.

The second right-foot measurement instrument 531 is attached to the right foot of the first user 11 or the right-foot shoe 21. Note that the second right-foot measurement instrument 531 may be included in a sole of the right-foot shoe 21.

The second left-foot measurement instrument 532 is attached to the left foot of the first user 11 or the left-foot shoe 22. Note that the second left-foot measurement instrument 532 may be included in a sole of the left-foot shoe 22.

The GNSS unit 551 is carried by the first user 11.

The second information processing device 541 may be, for example, a server device, and may have a function similar to the server device 52 illustrated in FIG. 4.

The second information processing device 541 is connected to a network. Note that in the embodiment, illustration of the network is omitted. The network may be a public network such as the Internet or a dedicated network.

The second information processing device 541 may be provided to any location.

In the embodiment, as compared to the configuration illustrated in FIG. 1, schematically, the mobile terminal device 51 illustrated in FIG. 1 is not provided, communication is performed between the second information processing device 541 and each of the second right-foot measurement instrument 531 and the second left-foot measurement instrument 532, and communication is performed between the second information processing device 541 and the GNSS unit 551.

These communications may be performed, for example, via a base station device connected to the network. In this case, radio communication is performed between the base station device and each of the second right-foot measurement instrument 531, the second left-foot measurement instrument 532, and the GNSS unit 551, and communication is performed between the base station device and the second information processing device 541, and thereby, communication is performed between the second information processing device 541 and each of the second right-foot measurement instrument 531, the second left-foot measurement instrument 532, and the GNSS unit 551. Note that in the embodiment, illustration of the base station device is omitted.

As another example, radio communication may be directly performed between the second information processing device 541 and each of the second right-foot measurement instrument 531, the second left-foot measurement instrument 532, and the GNSS unit 551.

Here, the GNSS unit 551, which includes a function of the GNSS, receives a GNSS signal to acquire information based on the received signal, similarly to the terminal GNSS unit 215 in the mobile terminal device 51 illustrated in FIG. 3.

The GNSS unit 551 has a function to transmit the acquired information to the second information processing device 541.

Note that in the second information processing device 541, in a configuration in which the information from the GNSS unit 551 is not used, the GNSS unit 551 need not necessarily be included.

As described above, in the second information processing system 501 according to the embodiment, the communication is performed between the second information processing device 541 and each of the second right-foot measurement instrument 531 and the second left-foot measurement instrument 532 without via the mobile terminal device.

Even with such a configuration, the effects similar to the first embodiment can be obtained.

Here, although the embodiments described above describe the case that the first user 11 runs like a marathon, a case that the first user 11 walks or a case that the first user 11 switches between walking and running may apply as another example.

Although the embodiments described above describes the case that both the right-foot measurement instrument corresponding to the right-foot shoe 21 and the left-foot measurement instrument corresponding to the left-foot shoe 22 apply as the measurement instrument, only one of these may apply, as another example.

Although the embodiments described above describes the case that the shoes used for running or the like apply, shoes for various sports such as golf, baseball, and tennis may apply, as another example.

Configuration examples of the embodiments described above are described.

According to a configuration example, an information processing system includes a measurement instrument and information processing device, and is configured as below.

The measurement instrument includes a data acquisition unit acquiring data about first shoes used by a user.

The measurement instrument or the information processing device includes a measurement result acquisition unit acquiring measurement results including at least a roll angle upon landing and a pitch angle upon landing, based on the data acquired by the data acquisition unit.

The information processing device includes a running motion determination unit performing at least determining a pronation score by comparison between the roll angle and a first threshold and determining a landing score by comparison between the pitch angle and a second threshold, and a suggestion determination unit determining suggested shoes, based on at least the pronation score and landing score.

Therefore, the information processing system can suggest the shoes suitable to the user.

Here, in the example in FIG. 1, the first information processing system 1 is an example of the information processing system. In the example in FIG. 1, each of the right-foot measurement instrument 31 and the left-foot measurement instrument 32 is an example of the measurement instrument. In the example in FIG. 1, the first information processing device 41 including the mobile terminal device 51 and the server device 52 is an example of the information processing device. In the example if FIG. 1, the first user 11 is an example of the user. In the example in FIG. 1, the first shoes 12 including the right-foot shoe 21 and the left-foot shoe 22 are examples of the first shoes.

In the example in FIG. 2, as for the right foot, the right-foot data acquisition unit 111 is an example of the data acquisition unit. Note that the same holds for the left foot.

In the example in FIG. 2, the right-foot data acquisition unit 111 may have a function of the measurement result acquisition unit.

In the example in FIG. 3, the terminal processing unit 216 may have the function of the measurement result acquisition unit.

In the example in FIG. 4, the first running motion determination unit 331 or the like in the first processing unit 315 may have the function of the measurement result acquisition unit.

In the example in FIG. 4, the first running motion determination unit 331 is an example of the running motion determination unit. In the example of FIG. 4, the first suggestion determination unit 332 is an example of the suggestion determination unit.

In the example in FIG. 17, the second information processing system 501 is an example of the information processing system. In the example in FIG. 17, each of the second right-foot measurement instrument 531 and the second left-foot measurement instrument 532 is an example of the measurement instrument. In the example in FIG. 17, the second information processing device 541 is an example of the information processing device.

According to a configuration example, in the information processing system, the pronation score includes a score for an overpronation and a score for an underpronation, and the landing score includes a score for a toe landing and a score for a heel landing. At least one of the score for the overpronation, the score for the underpronation, the score for the toe landing, and the score for the heel landing has a value in a plurality of stages.

Therefore, the information processing system can use the score having the a value in a plurality of stages to more accurately determine and suggest the shoes suitable to the user.

Note that a scheme for deciding each score may include various schemes.

According to a configuration example, in the information processing system, the measurement result further includes information about a running velocity. The running motion determination unit determines a running velocity score by comparison between the running velocity and a third threshold. The suggestion determination unit determines the suggested shoes, based on the pronation score, the landing score, and the running velocity score.

Therefore, the information processing system can use the running velocity to more accurately determine and suggest the shoes suitable to the user.

According to a configuration example, in the information processing system, the information processing device includes a manufacturer selection unit selecting a manufacturer. The suggestion determination unit selects the suggested shoes corresponding to shoes of the manufacturer selected by the manufacturer selection unit.

Therefore, the information processing system can select the manufacturer of shoes such that a preference of the user is reflected to suggest the shoes suitable to the user.

Here, in the example in FIG. 4, the first manufacturer selection unit 333 is an example of the manufacturer selection unit.

According to a configuration example, in the information processing system, the data acquisition unit includes an acceleration sensor and a gyroscope sensor.

Therefore, the information processing system can use the acceleration sensor and the gyroscope sensor to determine the pronation score and the landing score.

According to a configuration example, in the information processing system, the data acquisition unit includes a measurement instrument GNSS reception unit.

Therefore, the information processing system can use data of a position of the measurement instrument or data of time.

Here, in the example in FIG. 2, as for the right foot, the right-foot GNSS unit 133 has a function of the measurement instrument GNSS reception unit. Note that the same holds for the left foot.

According to a configuration example, in the information processing system, the information processing device includes an electronic device and a server device.

The measurement instrument includes a measurement instrument communication unit transmitting the acquired data or the measurement result to the electronic device.

The electronic device includes an electronic device communication unit receiving the data or the measurement result transmitted from measurement instrument and transmitting the data or the measurement result to the server device.

The server device includes a server communication unit receiving the data or the measurement result transmitted from the electronic device and transmitting information about the suggested shoes determined based on the data or the measurement result to the electronic device.

The electronic device receives, by the electronic device communication unit, the information about the suggested shoes transmitted from the server device.

The electronic device includes a display unit displaying the information about the suggested shoes received from the server device.

Therefore, the information processing system can display, by the electronic device, the information about the suggested shoes to the user.

Here, in the example in FIG. 1, the mobile terminal device 51 is an example of the electronic device. In the example in FIG. 1, the server device 52 is an example of the server device.

In the example in FIG. 2, as for the right foot, the right-foot communication unit 112 is an example of the measurement instrument communication unit. Note that the same holds for the left foot.

In the example in FIG. 3, the terminal communication unit 213 is an example of the electronic device communication unit.

In the example in FIG. 4, the first communication unit 313 is an example of the server communication unit.

In the example of FIG. 3, the terminal output unit 212 has a function of the display unit.

According to a configuration example, in the information processing system, the electronic device includes an electronic device GNSS reception unit. The data or the measurement result transmitted from the electronic device communication unit to the server device includes information based on a signal acquired by the electronic device GNSS reception unit.

Therefore, the information processing system can use data of a position of the electronic device or data of time.

Here, in the example in FIG. 3, the terminal GNSS unit 215 has a function of the electronic device GNSS reception unit.

The information processing device can be also provided.

According to a configuration example, the information processing device includes a measurement result acquisition unit acquiring measurement results including at least a roll angle upon landing and a pitch angle upon landing, based on an acquisition result of data about first shoes used by a user, a running motion determination unit performing at least determining a pronation score by comparison between the roll angle and a first threshold and determining a landing score by comparison between the pitch angle and a second threshold, and a suggestion determination unit determining suggested shoes, based on at least the pronation score and landing score.

Here, although the example in FIG. 1 illustrates an example in which the functions of the first information processing system 1 are distributively included in the right-foot measurement instrument 31 and the left-foot measurement instrument 32, the mobile terminal device 51, and the server device 52, the functions of the first information processing system 1 may be distributively included in two more devices according to any aspect.

For example, as in the example in FIG. 1, when the functions of the first information processing system 1 are distributively included in the right-foot measurement instrument 31 and the left-foot measurement instrument 32, the mobile terminal device 51, and the server device 52, in the configuration in which the data acquired by the right-foot measurement instrument 31 and the left-foot measurement instrument 32 is transmitted to the first information processing device 41, the right-foot measurement instrument 31 and the left-foot measurement instrument 32 may perform processing on the acquired data until any stage and thereafter, transmit the data to the first information processing device 41. Specifically, when the data acquired by the right-foot measurement instrument 31 and the left-foot measurement instrument 32 is processed by the right-foot measurement instrument 31 and the left-foot measurement instrument 32, and the first information processing device 41, the stage where the data is processed by the right-foot measurement instrument 31 and the left-foot measurement instrument 32 may be any stage, and the processing after the stage is performed by the first information processing device 41. In the first information processing device 41, the processing may be also arbitrarily distributed between the mobile terminal device 51 and the server device 52.

Similarly, although the example in FIG. 17 illustrates an example in which the functions of the second information processing system 501 are distributively included in the second right-foot measurement instrument 531 and the second left-foot measurement instrument 532, and the second information processing device 541, the functions of the second information processing system 501 may be distributively included in two more devices according to any aspect.

Although the embodiments described above describe the case that the functions of the information processing system are distributively included in two or more devices, the information processing system may be configured as an integrated system in another example. Such integrated system may be configured as a measurement instrument, and, for example, the measurement instrument may have functions to perform the whole processing performed in the information processing system according to the embodiments described above. Note that in such a configuration also, for example, the measurement instrument may be separately configured as the right-foot measurement instrument and the left-foot measurement instrument.

A program for implementing the function of any of the components in any of the devices described above may be recorded on a computer-readable recording medium to cause a computer system to read and execute the program. The “computer system” referred to herein includes an operating system or hardware such as peripheral devices. The “computer-readable recording medium” refers to a portable medium such as a flexible disk, a magneto-optical disc, a Read Only Memory (ROM), or a Compact Disc (CD)-ROM, a storage device such as a hard disk built in the computer system, or the like. The “computer-readable recording medium” includes those retaining the program for a certain time, like a volatile memory in the computer system that is to be a server or a client when a program is transmitted via a network such as the Internet or a communication line such as a telephone line. The volatile memory may be a RAM. The recording medium may be a non-transitory recording medium.

The program described above may be transmitted from the computer system storing the program in a storage device or the like to another computer system via a transmission medium or a transmission wave in the transmission medium. The “transmission medium” transmitting the program refers to a medium having a function to transmit information, like a network such as the Internet or a communication line such as a telephone line.

The above program may include those for implementing part of the functions described above. The above program may include those capable of implementing the functions described above in combination with a program already recorded in the computer system, that is, a so-called difference file. The difference file may be referred to as a difference program.

The function of any of the components in any of the devices described above may be implemented by a processor. Each processing in the embodiments may be implemented by a processor operating based on information such as a program and a computer-readable recording medium storing information such as a program. The processor may include units of which each of functions is implemented by dedicated hardware, or units of which functions are implemented by hardware integrated into one body. The processor includes hardware, and the hardware may include at least one of a circuit processing digital signals and a circuit processing analog signals. The processor may be configured using one or both of one or more circuit devices mounted at a printed wired board and one or more circuit elements. The circuit devices may include an Integrated Circuit (IC) or the like, and the circuit elements may include a resistor, a capacitor, or the like.

The processor may be a CPU. However, the processor is not limited to the CPU, and may include various processors such as a Graphics Processing Unit (GPU) or a Digital Signal Processor (DSP). The processor may include a hardware circuit using an Application Specific Integrated Circuit (ASIC). The processor may be configured to include a plurality of CPUs, or a plurality of hardware circuits using ASICs. The processor may be configured in combination of a plurality of CPUs and a plurality of hardware circuits using ASICs. The processor may include one or more of an amplifier circuit and filter circuit processing analog signals.

Although the embodiments of the present disclosure have been described above in detail with reference to the drawings, the specific configurations thereof are not limited to those of the embodiments and also include designs or the like without departing from the spirit of the present disclosure.

What is claimed is:

1. An information processing system comprising:
a measurement instrument; and
an information processing device, wherein
the measurement instrument includes a data acquisition unit configured to acquire data about first shoes used by a user,
the measurement instrument or the information processing device includes a measurement result acquisition unit configured to acquire a measurement result including at least a roll angle upon landing and a pitch angle upon landing, based on the data acquired by the data acquisition unit, and
the information processing device includes
a running motion determination unit configured to perform at least determination of a pronation score by comparison between the roll angle and a first threshold and determination of a landing score by comparison between the pitch angle and a second threshold and
a suggestion determination unit configured to determine suggested shoes, based on at least the pronation score and landing score.

2. The information processing system according to claim 1, wherein
the pronation score includes a score for an overpronation and a score for an underpronation,
the landing score includes a score for a toe landing and a score for a heel landing, and
at least one of the score for the overpronation, the score for the underpronation, the score for the toe landing, and the score for the heel landing has a value in a plurality of stages.

3. The information processing system according to claim 1, wherein
the measurement result further includes information about a running velocity,
the running motion determination unit is configured to determine a running velocity score by comparison between the running velocity and a third threshold, and
the suggestion determination unit is configured to determine the suggested shoes, based on the pronation score, the landing score, and the running velocity score.

4. The information processing system according to claim 1, wherein
the information processing device includes a manufacturer selection unit configured to select a manufacturer, and
the suggestion determination unit is configured to select the suggested shoes corresponding to shoes of the manufacturer selected by the manufacturer selection unit.

5. The information processing system according to claim 1, wherein
the data acquisition unit includes an acceleration sensor and a gyroscope sensor.

6. The information processing system according to claim 1, wherein
the data acquisition unit includes a measurement instrument GNSS reception unit.

7. The information processing system according to claim 1, wherein
the information processing device includes an electronic device and a server device,
the measurement instrument includes a measurement instrument communication unit configured to transmit the acquired data or the measurement result to the electronic device,
the electronic device includes an electronic device communication unit configured to receive the data or the measurement result transmitted from the measurement instrument and transmit the data or the measurement result to the server device,
the server device includes a server communication unit configured to receive the data or the measurement result transmitted from the electronic device and transmit information about the suggested shoes determined based on the data or the measurement result to the electronic device,
the electronic device is configured to receive, by the electronic device communication unit, the information about the suggested shoes transmitted from the server device, and
the electronic device includes a display unit configured to display the information about the suggested shoes received from the server device.

8. The information processing system according to claim 7, wherein
the electronic device includes an electronic device GNSS reception unit, and
the data or the measurement result transmitted from the electronic device communication unit to the server device includes information based on a signal acquired by the electronic device GNSS reception unit.

9. An information processing device comprising:
a measurement result acquisition unit configured to acquire a measurement result including at least a roll angle upon landing and a pitch angle upon landing, based on an acquisition result of data about first shoes used by a user;
a running motion determination unit configured to perform at least determination of a pronation score by comparison between the roll angle and a first threshold and determination of a landing score by comparison between the pitch angle and a second threshold; and
a suggestion determination unit configured to determine suggested shoes, based on at least the pronation score and landing score.
